(19) United States
(12) Patent Application Publication
(10) Pub. No.: US 2005/0153423 A1
(43) Pub. Date: Jul. 14, 2005

(54) Appliance for recovering solid component in liquid sample

(75) Inventors: Shigeaki Baba, Hyogo (JP); Tomiko Baba, Hyogo (JP); Takayuki Taguchi, Hyogo (JP); Toshiro Katayama, Hyogo (JP); Yasuro Kameno, Hyogo (JP); Shinichi Kameno, Hyogo (JP)

Correspondence Address: MARSHALL, GERSTEIN & BORUN LLP, 233 S. WACKER DRIVE, SUITE 6300, SEARS TOWER, CHICAGO, IL, 60606, US

(73) Assignee: HEALTH DYNAMICS INSTITUTE, Hyogo (JP)

(21) Appl. No.: 10/482,623

(22) PCT Filed: Mar. 20, 2002
(86) PCT No.: PCT/JP02/02635

(30) Foreign Application Priority Data
Jul. 2, 2001 (JP) 2001-200476

(51) Int. Cl. C12M 1/34; B01D 37/00

(52) U.S. Cl. 435/287.1; 210/768

(57) ABSTRACT

An assembled unit for collecting solid substance is provided for quickly and accurately collecting the solid substance in a liquid sample. The liquid sample introduced with suction is separated according to solid-liquid separation by the filter means 2 and the solid substance so separated is kept hermetically in the filter means 2. The solid substances are collected by applying pneumatic pressure from the inner side of the unit toward the filter matrix 3 and drifting the solid substances from the conduit 4, wherein the press means 13 is inserted into the support member 14 connected to the filter means 2.

### TECHNICAL FIELD

The present invention relates to an assembled unit for collecting solid substance from a liquid sample and to a method for collecting such solid substances with the assembled unit.

### BACKGROUND ART

Conventionally, in the clinical analysis for samples, in particlular, the solid samples (hereinafter simply referred to as “solid substance(s)”) taken from biological sources, they have generally been prepared by applying collected specimens to a physical process like centrifugation.

For example, when human urine was going to be analyzed, precipitate thereof was prepared as a solid substance by centrifuging fresh urine taken from the subject, then removing the supernatant so made and subjecting the remained solid to an analysis. Such precipitate is then analyzed microscopically on the presence of hemacytes, epithelial cells, cylindroids, salts, protozoa, bacteria and so on in the subjected sample (specimen), otherwise, they are incubated for determining the presence therein on bacteria and fungi.

Conventionally, raw liquids like drinkable water, tap water, sewage, industrial waste water, seawater, river water, lake water and so on were carried in an analysis organization, and were centrifuged for separating and collecting the solid substances in the raw liquids.

Accordingly, it was a routine practice in the conventional analysis on solid substance to apply a raw liquid to a separation process like centrifugation. Though a part of such separation process may be automated, considerable labor and skill are necessary to perform it, and the necessary labor will be larger synergistically according to an increase of sample number to be analyzed.

Further, raw liquid often contains some components that will easily be substantially changed by bacteria, oxygen, light or the like and, in order to realize accurate analysis, such samples are therefore usually preserved under frozen or refrigerated condition until the analysis. In particular, when the large number of samples are going to be analyzed in a short time, the conventional art relied on an ineffective method comprising the steps of hermetically packing samples, then preserving them under frozen (refrigerated) condition, and thawing the same at the analyzing of them.

The art needed quick and reliable separation process to collect solid substances from raw liquid, nevertheless, the prior arts aforenoted have not yet fairly responded to such demands in the art.

### DISCLOSURE OF INVENTION

The present invention has been established in view of the aforenoted problems in the prior art, and the merit of the present invention is directed to an assembled unit for collecting solid substance from liquid samples (raw liquid samples) which comprises a path that connects outside of the unit with inside thereof and a filter means that keeps solid substance remained by separating, according to solid-liquid separation, the liquid samples introduced thereinto through the path.

The filter means have a filter matrix at the inside thereof for separating, according to solid-liquid separation, the liquid sample. In order to prevent a proliferation of microorganisms lived spontaneously in the filter matrix, a sterilized filter matrix is preferable as the filter matrix. For example, when a liquid sample containing bacteria is subjected to a filtration, in order to exclude such bacteria and to allow smooth flow of unnecessary liquid phase, it is preferable to employ a filter matrix like that having taper holes or that having a pore diameter of from about 0.05 μm to about 0.2 μm.

The assembled unit of the present invention further comprises press means for drifting the solid substance in the liquid sample (raw liquid sample) introduced into the filter means to the outside thereof.

Under such situation, the assembled unit of the present invention further comprises preferably a support member which can be connected to the filter means and can receive the press means to be inserted thereinto. If a tubular support member is employed, for example, a press means bar is utilized appropriately.

Since the solid substances, which is separated according to solid-liquid separation from the liquid sample and is remained in the assembled unit of the present invention, are isolated from outside environment of the unit containing bacteria, oxygen, light and so on, substantial change of the solid substances can effectively be obviated, then the solid substances can be preserved for the longer term without the frozen/refrigerated condition.

Further, according to the assembled unit of the present invention, solid substances remained in the assembled unit are drifted to the outside of the filter means by entering gas and/or applying pneumatic pressure from a suction outlet of the filter means. Otherwise, solid substances remained in the assembled unit are drifted to the outside of the filter means by entering liquid like water and/or applying pressure from a suction outlet of the filter means.

As stated above, according to the present invention, the solid substances in the liquid samples can be separated and be collected without any centrifugation, and necessary labor force for preparing a test sample will also be reduced thereby.

### BRIEF DSEXCRIPTION OF DRAWINGS

FIG. 1 is a partially cutaway perspective view of the filter means.

FIG. 2 is a perspective view of the filter means.

FIG. 3 is an exploded perspective view illustrating one example of the assembled unit of the present invention.

FIG. 4 is a perspective view illustrating one example of the assembled unit of the present invention.

FIG. 5 is an exploded perspective view illustrating one example of the press means in the assembled unit of the present invention.

FIG. 6 is an explanatory view illustrating one example on the process to collect the solid substance with the assembled unit of the present invention.

FIG. 7 is an explanatory view illustrating another example on the process to collect the solid substance with the assembled unit of the present invention.

### BEST MODE FOR CARRYING OUT THE INVENTION

The present invention will be described in detail as follows.

First, the term “liquid sample” employed herein is used synonymously with the term raw liquid and includes any liquid taken from biological fluids like blood, ascites, dialysate, urine, cerebrospinal fluid, pus, sputum, semen, and any liquid taken from environmental waters like drinkable water, tap water, sewage, industrial waste water, seawater, river water, lake water.

The assembled unit of the present invention essentially comprises a path that connects outside of the unit to a filter means that keeps solid substance remained by separating, according to solid-liquid separation, the liquid samples introduced thereinto through the path.

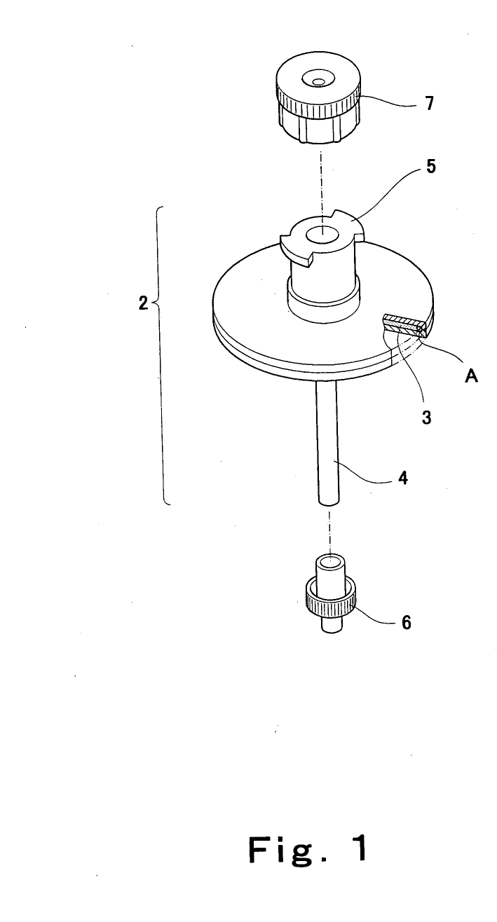

With reference to FIG. 1, the filter means 2 has a filter matrix 3 in its internal space A, and the conduit 4 is equipped at the inlet side where the raw liquid is sucked at the collecting thereof while a connector portion 5 is equipped at the opposite outlet side. When the raw sample is not taken, the stoppers 6 and 7 are engaged respectively with the conduit 4 and the connector portion 5. Thereby, the stoppers 6 and 7 block the communication between the internal space of the filter means 2 and the outside environment.

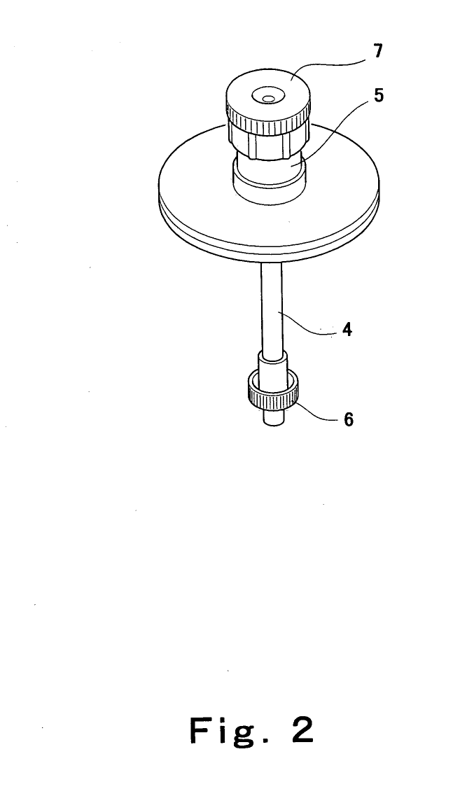

Thus, in order to collect the raw sample, the stoppers 6 and 7 are firstly detached from the filter means 2, then suction means (not shown) connected to the connector portion 5 is driven to reduce the pressure in the filter means 2, and the liquid sample is guided to the outlet side through the conduit 4, filter matrix 3 and connector portion 5. Thereby, the liquid sample introduced into the filter means 2 is separated immediately according to solid-liquid separation into a solid substance and a liquid phase with the filter matrix 3 put into the filter means 2, and the solid substance is kept in the filter means 2. Subsequently, the stoppers 6 and 7 are engaged respectively with the conduit 4 and the connector portion 5 (as illustrated in FIG. 2), then the collected samples are hermetically preserved until use.

As the filter matrix 3, it should preferably be made of cellulose acetate, polysulfone and polyether sulfone, each of which has less adsorbability on the solid substances (e.g., protein). Then in view of to prevent proliferation of microorganisms spontaneously lived in the filter matrix 3, a sterilized filter matrix is preferable. Further, in order to remove bacteria from the filter matrix 3 and to allow smooth flow of unnecessary liquid phase, it is preferable to employ a filter matrix 3 as that having taper holes or that having a pore diameter of from about 0.05 μm to about 0.2 μm.

Conduit 4 and connector portion 5 in the filter means 2 can be made respectively from materials like glass and synthetic resin, and flexibility and size of each of them can also be appropriately adjusted. Alternatively, as a matter of course, any part in the filter means 2 other than the filter matrix 3 may also be made from the same material.

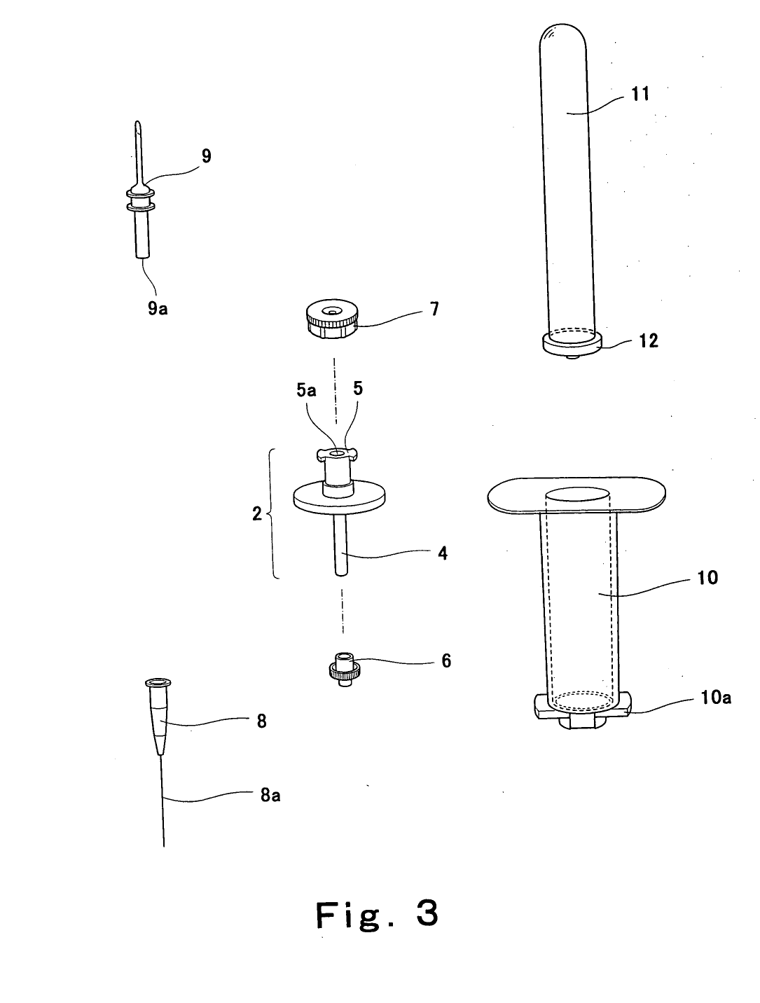

FIG. 3 illustrates cardinal elements in the assembled unit of the present invention. Of these elements, the assembled unit of the present invention essentially includes the filter means 2 that keeps solid substance remained by separating, according to solid-liquid separation, the liquid samples introduced thereinto.

In accordance with a preferred embodiment of the assembled unit of the present invention, a tip 8 equipped with a needle 8a having an longitudinally extended internal hollow is engaged with the conduit 4 of the filter means 2 to collect the liquid phase of the liquid sample. Though the liquid sample can directly be collected with the conduit 4 without the tip 8, the tip 8 enables a person to collect trace amount of the liquid sample or to collect the liquid sample from an intricate tissues. Meanwhile, commercially available non-bevel needle can advantageously be utilized in the present invention as the tip 8.

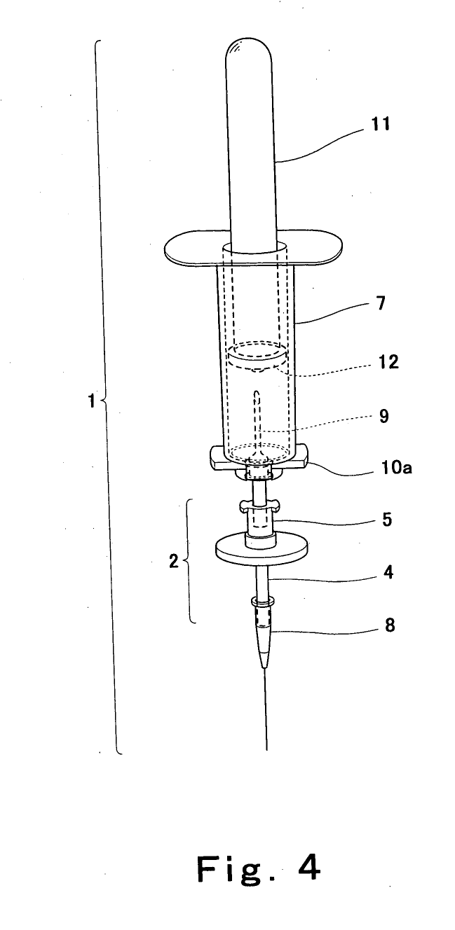

Then, a coupler 9 is attached to the filter means 2 by inserting a suction tube 9a of the coupler 9, which has an longitudinally extended internal space therein, into a port 5a formed in the connector 5 of the filter member 2. The filter means 2 is then coupled to a supporter 10 by engaging the lower end 10a of the supporter 10 with the connector 5. According to this embodiment, the assembled unit of the present invention is preferably equipped with a collection container 11 (to be acted as suction means) which can be inserted into the supporter 10 and can receive the liquid phase of the liquid sample to be introduced. With reference to FIG. 4, the assembled unit 1 of the present invention is consisting of these elements aforenoted.

Materials and structure of the container 11 may be appropriately selected/designed in order to, for example, block the communication of air between the inside and the outside of the container 11, then the container 11 may be filled once with an inert gas like nitrogen gas to realize a reduced pressure and hermetically be closed with a stopper 12 made of synthesized rubber. Though the container 11 needs reduced pressure which enables it to collect thereinto a sufficient amount of the liquid phase of the liquid sample, an excessive pressure reduction should be avoided in view of to effectively keep an appropriate pressure and the durability of the container, and the pressure is usually adjusted to the range of from about 0.1 to about 0.5 atm as long as the container is used under normal atmospheric pressure, though it depends on the material of the container 11. Size and shape of the container 11 is preferably adjusted/formed to become those like a test tube or bottle which may allow to be attached it to a popular analyzer, there is no particular limitation however as long as the container 11 can be inserted into the supporter 10, preferably in a manner that a sharpened head of the coupler 9 engaged with the inner end of the supporter 10 pierces the stopper 12. By shading the container 11, substantial property of the sample can hardly be changed due to light.

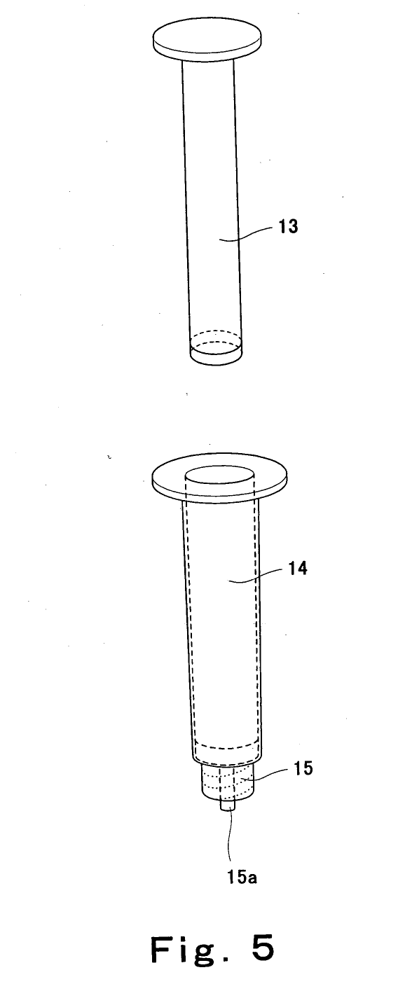

The assembled unit of the present invention further comprises a press means, cardinal elements of which are illustrated in FIG. 5, to remove the solid substance from the filter means to outside of the unit. Any means which can drifted the solid substance from the filter means 2 to outside of thereof are applicable as the pressing means, and it may includes a means to apply compressed air to the filter means 2 and that to continuous supply water to the filter means 2. In the press means having the elements illustrated in FIG. 5, the press means is a combination of the press means 13 and a support member 14 which will be coupled to the filter means 2 and can receive the press means 13 to be inserted thereinto. Any structure would be employed for the press means 13 as long as it can be inserted into the support member 14, but it is preferable to employ a structure which allow to insert it into the support member 14 without losing sealablity in the support member 14. For example, with reference to FIG. 4, press means bar 13 is usually used in combination with tubular support member 14. It is possible to make the support member 14 and the press means 13 respectively from individual materials like glass and synthetic resin and to adjust appropriately their flexibility. Alternatively, both the support member 14 and the press means 13 may be formed from the same material.

Any inner diameter of the press means 13 is applicable as long as it can be inserted into the support member 14 without losing sealablity in the support member 14. Then the filter means 2 is coupled to the support member 14 by engaging the connector portion 5 in the filter means 2 with the groove 15 tapped at an internal surface of the coupling portion at the lower end of a support member 14. When the connector portion 5 is engaged with the groove 15, the drain tube 15a is simultaneously engaged with the outlet 5a, thereby, inside of the support member 14 and that of the filter means 2 are communicated.

Procedure as to how to use the assembled unit of the present invention is as follows.

With reference to FIG. 1, the stoppers 6 and 7 are detached from the filter means 2, then the suction means (not shown) connected to the connector portion 5 in the filter means 2 is driven to reduce the atmospheric pressure in the filter means 2, thereby, the liquid sample is guided toward the connector portion 5 in the filter means 2 through the conduit 4 and the filter means 2. The solid substance of the liquid sample introduced in the filter means 2 is separated by the filter matrix 3 put in the filter means 2 and is kept within the filter means 2.

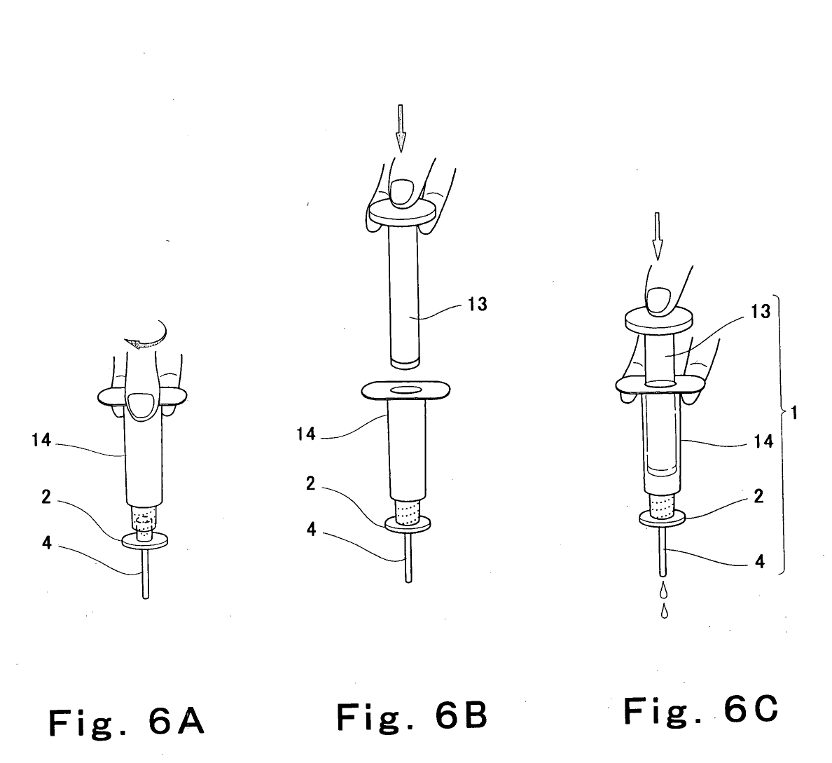

With reference to FIG. 6, in order to collect the solid substance kept in the filter means 2 of the assembled unit 1 of the present invention, firstly, the connector portion 5 is engaged with the groove 15 tapped at the internal surface of the coupling portion of the support member 14 (FIG. 5), thereby, the filter means 2 is coupled to the support member 14 (FIG. 6(a)). When the press means 13 is then inserted into the support member 14, pneumatic pressure from the suction outlet of the filter means 2 is applied to the filter matrix 3, and the solid substance is drifted into the conduit 4 (FIGS. 6(b) and 6(c)). Such solid substance is collected in an appropriate container (e.g., a test tube with stopper) to hermetically preserve the same.

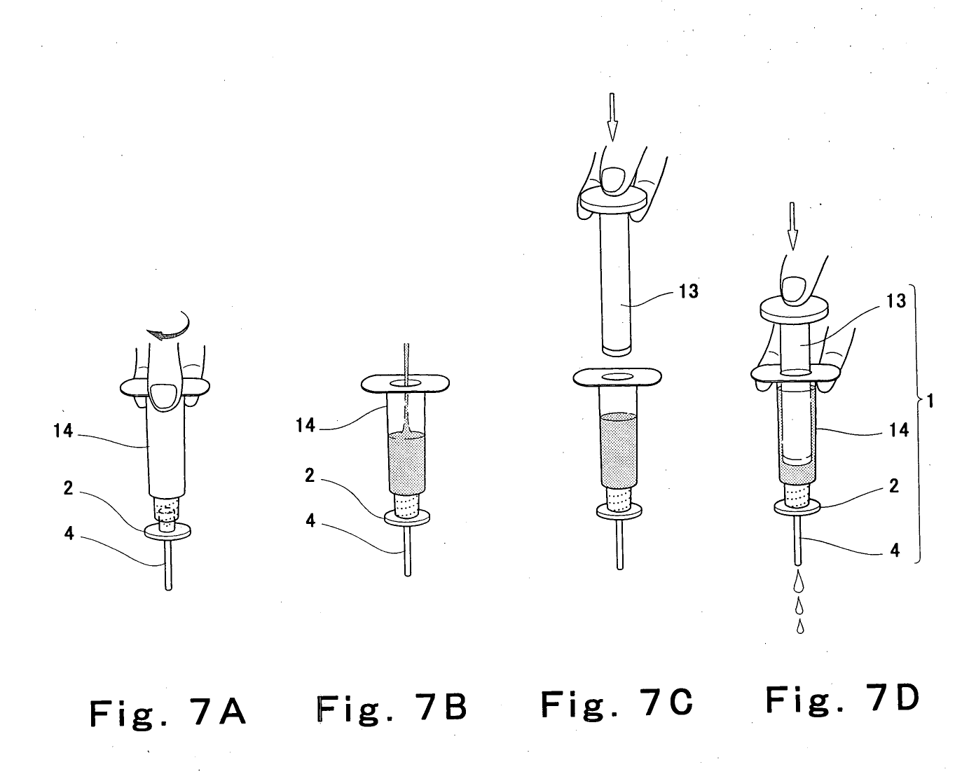

Alternatively, in accordance with another embodiment to collect the solid substance kept in the filter means 2 of the assembled unit 1, hydraulic pressure like water pressure is employed. With reference to FIG. 7, for example, firstly, the connector portion 5 is engaged with the groove 15 (FIG. 5) tapped at the internal surface of the coupling portion in the support member 14, thereby, the filter means 2 is coupled to the support member 14 (FIG. 7(a)). Subsequently, a liquid such as water is poured into the support member 14 (FIG. 7(b)), then the press means 13 is inserted into the support member 14, thereby, water pressure (liquid pressure) from the suction outlet side of the filter means 2 is applied to the filter matrix 3, and the solid substance retained in the filter matrix 3 of the filter means 2 is drifted to the conduit 4 together with the poured liquid (FIGS. 7(c) and 7(d)). Any liquid is applicable as long as it may not change the substantial properties of the solid substance and may includes, for example, deionized water, sterilized water, distilled water, purified water, physiologic saline and low-concentration formalin solution. If a liquid culture medium is used, the solid substance and the liquid medium can simultaneously be removed from the filter means 2, thereby, bacteria and the like kept alive until the analysis. Alternatively, if water is used to remove the solid substance from the filter means 2, bacteria and the like would also be kept alive until the analysis.

Such solid substance is collected in an appropriate container (e.g., a test tube with stopper) to hermetically preserve the same. Amount of the liquid to be drifted from the conduit 4 can be adjusted by changing the amount of the liquid (water) to be poured into the support member 14.

The present invention will be described in detail according to the following examples, but the present invention should not be interpreted based on the disclosures of the illustrative examples.

### EXAMPLE 1

First of all, the following samples were provided as liquid samples.
Sample 1: Sewage (Households Sewage)
Sample 2: River Water (from Fukuda River in Tarumi, Kobe)
Sample 3: Tap Water (from water-works in a collective housing)
Sample 4: Tap Water (from water-works in a school)
Sample 5: Industrial Waste Water (from a laundry)
Sample 6: Mineral Water (commercial name: EVIAN)
Sample 7: Green Tea (commercial name: NAMACHA)
Sample 8: Fresh Urine (taken from a healthy adult female)
Sample 9: Fresh Urine (taken from a healthy adult female)

Filter means was then provided wherein it had a filter matrix in its internal space, and the conduit was equipped at the inlet side where the raw liquid was sucked at the collecting thereof while a connector portion was equipped at the opposite outlet side. Next, the stoppers were removed from open ends of both the conduit and the connector portion, then a suction tube of a coupler was engaged with a port formed in the connector portion of the filter means in order to attach the coupler to the filter means. Subsequently, the lower end of the supporter was engaged with the connector portion in order to couple the filter means to the supporter. Thereafter, the container for collecting a liquid phase was inserted into the supporter to a position where the stopper was just reached to a sharpened head of the coupler.

Samples 1-9 were respectively collected into cups. After immersing the individual conduit into each of the samples, the container was further inserted toward the lower end of the supporter, so that the sharpened head of the coupler pierced the stopper. Though a tip was not engaged with the coupler according to this example, it is possible to optionally use such tip according to the judgment by the operator.

Thus, reduced pressure in the sealed container guided the liquid sample in each cup to the filter means through the conduit. The liquid sample introduced in the filter means was separated according to solid-liquid separation into a solid substance and a liquid phase, and the solid substance was kept in the filter means. Thereafter, the container containing the liquid phase was removed from the supporter and was preserved as it was at room temperature.

As the filter matrix, the filter matrix (sterilized; 50 mm diameter) had been employed in which the matrix was made of cellulose acetate and had taper holes in a diameter of 0.2 μm. Then, as the container, a brown plastic test tube had been employed in which the tube was hermetically closed with a rubber stopper and the internal pressure thereof was reduced to the range from −50 kPa to −80 kPa.

In order to collect the solid substance, first of all, the supporter and the coupler were detached from the filter means. Then the filter means was coupled to the support member by engaging the connector portion in the filter means with the groove tapped at an internal surface of the coupling portion of a support member. At that time, the drain tube of the support member was engaged with the outlet of the filter means, thereby, inside of two elements are communicated therebetween. Purified water was poured into the support member which is coupled to the filter means. Press means bar was inserted into the tubular support member containing the purified water so poured, the solid substance in the filter means was then discharged from the conduit together with the purified water, and the discharged liquid was collected into a plastic test tube.

The raw liquid sample of Samples 1-9 as well as both the liquid phase and the solid substance respectively collected from each of them according to the aforenoted procedure were measured with regard to pH, chemical oxygen demand (COD), urinoglucose and albuminuria, and the presence of bacteria.

pH of the samples were measured with pH INDICATOR (commercial name, Whatman Co.).

COD in the samples were measured with a COD measurement kit (water quality pack test; KYORITSU RIKAGAKU KENKYUSHO Co. Ltd.).

Urinoglucose and albuminuria in the samples were determined with ex vivo diagnostic reagent pretest (Wako Pure Chemical Industries, Ltd.).

The presence of bacteria in the samples was confirmed by utilizing a solid culture medium and a liquid culture medium.

Specifically, as a solid culture medium, LB medium [peptone (10 g), yeast extract (5 g), sodium chloride (5 g) and agar (15 g) per 1 liter of the medium] was prepared. 20 ml of the medium was applied to each plate, then 50 l of each liquid sample was applied thereto, and the culture was allowed to grow statically at room temperature followed by evaluation on colony formation on the medium, namely, the presence of the bacteria.

Similarly, as a liquid culture medium, FT medium [Bactocasitone (15 g), Bactoyeast extract (5 g), Bactodextrose (5.5 g), sodium chloride (2.5 g), L-cysteine (0.5 g), sodium thioglycolate (0.5 g), Bactoagar (0.75 g) and resazurin (0.001 g) per 1 liter of the medium] was prepared. 10 ml of this medium was poured into each test tube, then 0.5 ml of each liquid sample was added to the medium, and the culture was allowed to grow statically at room temperature followed by evaluation on turbidity of the medium, namely, the presence of the bacteria.

Measurement results on the liquid samples are shown in the following Table 1.

TABLE 1

| | | | COD | CULTIVATION | CULTIVATION |
|---|---|---|---|---|---|
| | | PH | (ppm) | (LB MEDIUM) | (FT MEDIUM) |
| Sample 1 | Raw Liquid | 6.8-7.0 | 10-20 | Colonies Appeared | turbid meidum |
| | Solid Substances | 6.8-7.0 | 10-20 | Colonies Appeared | turbid medium |
| | Liquid Phases | 6.8-7.0 | 10-20 | No Changed | No Changed |
| Sample 2 | Raw Liquid | 6.8-7.0 | 0-10 | Colonies Appeared | turbid meidum |
| | Solid Substances | 6.8-7.0 | 0-10 | Colonies Appeared | turbid medium |
| | Liquid Phases | 6.8-7.0 | 0-10 | No Changed | No Changed |
| Sample 3 | Raw Liquid | 6.5-6.8 | 0-10 | No Changed | No Changed |
| | Solid Substances | 6.5-6.8 | 0-10 | No Changed | No Changed |
| | Liquid Phases | 6.5-6.8 | 0-10 | No Changed | No Changed |
| Sample 4 | Raw Liquid | 6.5-6.8 | 0-10 | No Changed | No Changed |
| | Solid Substances | 6.5-6.8 | 0-10 | No Changed | No Changed |
| | Liquid Phases | 6.5-6.8 | 0-10 | No Changed | No Changed |
| Sample 5 | Raw Liquid | 8.1< | 100< | Few Colonies Appeared | Slightly turbid meidum |
| | Solid Substances | 8.1< | 100< | Colonies Appeared | turbid medium |
| | Liquid Phases | 8.1< | 100< | No Changed | No Changed |
| Sample 6 | Raw Liquid | 7.0-7.2 | 0 | No Changed | No Changed |
| | Solid Substances | 7.0-7.2 | 0 | No Changed | No Changed |
| | Liquid Phases | 7.0-7.2 | 0 | No Changed | No Changed |
| Sample 7 | Raw Liquid | 6.3-6.6 | 0 | No Changed | No Changed |
| | Solid Substances | 6.3-6.6 | 0 | No Changed | No Changed |
| | Liquid Phases | 6.3-6.6 | 0 | No Changed | No Changed |
| Sample 8* | Raw Liquid | 5.7-5.9 | Not Tested | No Changed | No Changed |
| | Solid Substances | 5.7-5.9 | Not Tested | No Changed | No Changed |
| | Liquid Phases | 5.7-5.9 | Not Tested | No Changed | No Changed |
| Sample 9* | Raw Liquid | 5.7-5.9 | Not Tested | No Changed | No Changed |
| | Solid Substances | 5.7-5.9 | Not Tested | No Changed | No Changed |
| | Liquid Phases | 5.7-5.9 | Not Tested | No Changed | No Changed |

*Neither urinoglucose nor albuminuria was detected.

Apparent from the results in Table 1, there was no difference between the liquid phase and the solid substance in each sample with regard to determination results on pH, COD, urinoglucose and albuminuria. Further, regarding the liquid samples in which bacteria had been confirmed in both their raw liquids and the solid substances, any bacteria had not been confirmed in all of their liquid phases.

These results clearly indicated that the assembled unit of the present invention had exactly divided the liquid sample into the solid substance and the liquid phase.

### EXAMPLE 2

Based on the results in Example 1, urine taken from a patient who received dialysis (a patient suffering from chronic renal failure or the like) was used as a liquid sample, and the liquid phase thereof was collected similarly according to the procedure noted in Example 1. The collection of the solid substance was performed according to the following three methodologies.

Method 1: Immediately after the collection of the liquid phase, both the supporter and the coupler were detached from the filter means, and the connector portion in the filter means was engaged with the groove tapped at the internal surface of the coupling portion of the support member to connect the filter means to the support member. Then the press means bar was inserted into the support member to gradually apply pneumatic pressure to the filter means, and the liquid appeared in the conduit was fed into a plastic test tube.

Method 2: After the collection of the liquid phase, both the supporter and the coupler were detached from the filter means and the filter means was allowed to stand for a while, subsequently the connector portion in the filter means was engaged with the groove tapped at the internal surface of the coupling portion of the support member to connect the filter means to the support member. Then the press means bar was inserted into the support member to gradually apply pneumatic pressure to the filter means, and the liquid appeared in the conduit was fed into a plastic test tube.

Method 3: After the collection of the liquid phase, both the supporter and the coupler were detached from the filter means and the filter means was vibrated, subsequently the connector portion in the filter means was engaged with the groove tapped at the internal surface of the coupling portion of the support member to connect the filter means to the support member. Then the press means bar was inserted into the support member to gradually apply pneumatic pressure to the filter means, and the liquid appeared in the conduit was fed into a plastic test tube. The solid substances obtained by Methods 1-3 were subjected to microscopic analysis to count urine components (Erythrocyte, Epithelial Cells, Cylindroids) in each of the solid substances. Solid substances taken from the same sources and prepared by centrifugation (conventional method) were also subjected to microscopic analysis to count the urine components in the same manner as noted above. The results of such analyses are indicated in Table 2 below.

The microscopic analyses were conducted under a magnification of 400 times. The number on the solid substances collected according to Methods 1-3 are indicated as percentages determined based on the corresponding number according to the centrifugation which is assumed as 100.

TABLE 2

| | Centrifugation | | | |
|---|---|---|---|---|
| | Method | | | |
| | (Counted Number) | Method 1 | Method 2 | Method 3 |
| Erythrocyte | 100 (28) | 82 | 89 | 93 |
| Epithelial Cells | 100 (17) | 80 | 91 | 88 |
| Cylindroids | 100 (15) | 80 | 86 | 107 |

Apparent from the results in Table 2, there was no significant difference in the numbers counted with microscopic analysis between the conventional method and the methods according to the present invention.

These results clearly indicated that the assembled unit of the present invention has separated the solid substance from a liquid sample without changing substantial properties of the solid substance.

### INDUSTRIAL APPLICABILITY

As stated above, the assembled unit of the present invention can rapidly separate and collect the solid substance in a liquid sample at the collecting of the liquid sample (raw liquid sample).

Since the solid substance taken from the filter means in the assembled unit of the present invention can directly be used as a sample for a qualitative analysis and a cultivation test, centrifugation of the solid substance is no longer necessary according to the present invention, though the prior art still needs such centrifugation at the preparing of the sample.

Further, according to the assembled unit of the present invention, it is not necessary to preserve a separated solid substance under the frozen or refrigerated condition, considerable reduction on labor force and cost of facility for such preservation would therefore also be expected.

1. An assembled unit for collecting solid substance from liquid samples comprising a path that connects outside of the unit to a filter means that keeps solid substance remained by separating, according to solid-liquid separation, the liquid samples introduced thereinto through the path.

2. The assembled unit according to claim 1, wherein the filter means has a filter matrix that separates the liquid samples according to solid-liquid separation.

3. The assembled unit according to claim 2, wherein the filter matrix is sterilized.

4. The assembled unit according to claim 2, wherein the filter matrix has taper holes.

5. The assembled unit according to any of claim 2, wherein a pore diameter of the filter matrix is from about 0.05 μm to about 0.2 μm.

6. The assembled unit according to any of claim 1 further comprises a press means for drifting the solid substance in the liquid sample to the outside of the filter means.

7. The assembled unit according to claim 6 further comprises a support member which can be connected to the filter means and allow insertion of the press means thereinto.

8. A method for collecting solid substances in a liquid sample with the assembled unit according to claim 1 any comprising the steps of;
introducing the liquid sample into a filter means with suction;
separating the liquid sample according to solid-liquid separation; and
keeping the solid substance separated according to the solid-liquid separation in the filter means.

9. The method according to claim 8 further comprising the step of drifting the solid substance into the path of the filter means by a press means.

10. The method according to claim 9, wherein the solid substance is drifted together with a liquid medium.

11. The assembled unit according to claim 3, wherein the filter matrix has taper.

12. The assembled unit according to any of claim 3, wherein a pore diameter of the filter matrix is from about 0.05 μm to about 0.2 μm.

13. The assembled unit according to any of claim 4, wherein a pore diameter of the filter matrix is from about 0.05 μm to about 0.2 μm.

14. The assembled unit according to any of claim 2 further comprises a press means for drifting the solid substance in the liquid sample to the outside of the filter means.

15. The assembled unit according to any of claim 3 further comprises a press means for drifting the solid substance in the liquid sample to the outside of the filter means.

16. The assembled unit according to any of claim 4 further comprises a press means for drifting the solid substance in the liquid sample to the outside of the filter means.

17. The assembled unit according to any of claim 5 further comprises a press means for drifting the solid substance in the liquid sample to the outside of the filter means.

18. A method for collecting solid substances in a liquid sample with the assembled unit according to claim 2 comprising the steps of;
introducing the liquid sample into a filter means with suction;
separating the liquid sample according to solid-liquid separation; and
keeping the solid substance separated according to the solid-liquid separation in the filter means.

19. A method for collecting solid substances in a liquid sample with the assembled unit according to claim 3 comprising the steps of;
introducing the liquid sample into a filter means with suction;
separating the liquid sample according to solid-liquid separation; and
keeping the solid substance separated according to the solid-liquid separation in the filter means.

20. A method for collecting solid substances in a liquid sample with the assembled unit according to claim 4 comprising the steps of;
introducing the liquid sample into a filter means with suction;
separating the liquid sample according to solid-liquid separation; and
keeping the solid substance separated according to the solid-liquid separation in the filter means.

21. A method for collecting solid substances in a liquid sample with the assembled unit according to claim 5 comprising the steps of;
introducing the liquid sample into a filter means with suction;
separating the liquid sample according to solid-liquid separation; and
keeping the solid substance separated according to the solid-liquid separation in the filter means.

22. A method for collecting solid substances in a liquid sample with the assembled unit according to claim 6 comprising the steps of;
introducing the liquid sample into a filter means with suction;
separating the liquid sample according to solid-liquid separation; and
keeping the solid substance separated according to the solid-liquid separation in the filter means.

23. A method for collecting solid substances in a liquid sample with the assembled unit according to claim 7 comprising the steps of;
introducing the liquid sample into a filter means with suction;
separating the liquid sample according to solid-liquid separation; and
keeping the solid substance separated according to the solid-liquid separation in the filter means.
